SEQUENCE LISTING (1 sequence)

SEQ ID NO: 1 - protein, 551 aa, Homo sapiens
AGAGGACGTTTCCAACTCAAAAATGCAGGCTCAACAGTACCAGCAGCAGCGTCGAAAATTTGCAGCTGCCTTCTTGGCATTCATTTTCATACTGGCAGCTGTGGATACTGCTGAAGCAGGGAAGAAAGAGAAACCAGAAAAAAAAGTGAAGAAGTCTGACTGTGGAGAATGGCAGTGGAGTGTGTGTGTGCCCACCAGTGGAGACTGTGGGCTGGGCACACGGGAGGGCACTCGGACTGGAGCTGAGTGCAAGCAAACCATGAAGACCCAGAGATGTAAGATCCCCTGCAACTGGAAGAAGCAATTTGGCGCGGAGTGCAAATACCAGTTCCAGGCCTGGGGAGAATGTGACCTGAACACAGCCCTGAAGACCAGAACTGGAAGTCTGAAGCGAGCCCTGCACAATGCCGAATGCCAGAAGACTGTCACCATCTCCAAGCCCTGTGGCAAACTGACCAAGCCCAAACCTCAAGCAGAATCTAAGAAGAAGAAAAAGGAAGGCAAGAAACAGGAGAAGATGCTGGATTAAAAGATGTCACCTGTGGAACATA

(19) United States
(12) Patent Application Publication
(10) Pub. No.: US 2008/0039382 A1
(43) Pub. Date: Feb. 14, 2008

(54) Compositions related to pleiotrophin methods and uses thereof

(76) Inventors: Randall J. Lee, Hillsborough, CA (US); Kenneth J. Colley, San Francisco, CA (US)

Correspondence Address: Pepper Hamilton LLP, One Mellon Center, 50th Floor, 500 Grant Street, Pittsburgh, PA, 15219, US

(21) Appl. No.: 10/857,087

(22) Filed: May 28, 2004

Related U.S. Application Data
(60) Provisional application No. 60/474,028, filed on May 29, 2003.
(60) Provisional application No. 60/476,787, filed on Jun. 6, 2003.

(51) Int. Cl. A61K 38/16; A61K 31/7052; A61P 9/00; C12N 15/63

(52) U.S. Cl. 514/12; 435/320.1; 514/44

(57) ABSTRACT

The present invention relates to compositions and methods useful for stimulating or increasing angiogenesis and inducing repair in damaged or diseased tissue, in vivo. More particularly, the present invention is directed to compositions comprising pleiotrophin and methods of inducing and stimulating angiogenesis particularly in ischemic tissue and cardiovascular tissue. The present invention also provides vectors comprising pleiotrophin. These pleiotrophin vectors express pleiotrophin in vivo and are capable of inducing and stimulating angiogenesis in cardiovascular tissue.

This application claims priority to U.S. Provisional Application No. 60/474,028 filed May 29, 2003 and U.S. Provisional Application No. 60/476,787 filed Jun. 6, 2003, the contents of each are incorporated herein in their entirety.

### FIELD OF THE INVENTION

The present invention relates to compositions and methods useful for stimulating or increasing angiogenesis and inducing repair in damaged or diseased tissue, in vivo. More particularly, the present invention is directed to compositions comprising pleiotrophin and methods of inducing and stimulating angiogenesis, particularly, in cardiovascular tissue.

### BACKGROUND OF THE INVENTION

Cardiovascular disease is the most common cause of death and disability in the United States. Coronary heart disease affects millions of Americans, and arises from damage to the cardiac muscle, the myocardium, caused by insufficient flow of blood in the coronary arteries resulting in the reduced ability to deliver oxygen and nutrients to the heart muscle. The resulting heart damage is reflected in conditions such as myocardial infarction, angina, unstable angina, and sudden ischemic death as heart failure.

Ischemia is a condition characterized by a lack of oxygen supply in tissues of organs and affects cardiovascular tissue as demonstrated in ischemic heart disease. Ischemic heart disease is caused, primarily, by atherosclerosis of the coronary arteries. This condition is characterized by plaque formation in the inner lining of the arteries, causing narrowing of the channel and thereby impairing blood flow to the heart. More particularly, myocardial ischemia is characterized, for example, by an imbalance between the myocardial blood flow and the metabolic demand of the myocardium.

Ischemia occurs when a tissue receives an inadequate supply of blood. For example, myocardial ischemia occurs when cardiac muscle does not receive an adequate blood supply. This can be due to occlusion or narrowing of the blood vessels, such as seen in coronary artery atherosclerosis. Treatments include surgical and pharmaceutical approaches. Surgical intervention is used to widen the narrowed lumens (e.g., balloon angioplasty) or to increase the numbers of cardiac blood vessels (e.g., bypass surgery using grafts). Less traumatic pharmaceutical treatments act to decrease cardiac muscle demand for oxygen and nutrients or to increase the blood supply. Oxygen demand can be lowered by decreasing the contractile response of the heart to a hemodynamic load (e.g., using beta-adrenergic blockers).

As many as 1.5 million patients per year in the U.S. suffer a myocardial infarction. Many millions more suffer from syndromes of chronic myocardial ischemia due to large and small vessel coronary atherosclerosis. Acute myocardial infarction (AMI) results from the rapid development of myocardial necrosis caused by a critical imbalance between the oxygen supply and demand of the myocardium. This usually results from plaque rupture with thrombus formation in a coronary vessel, resulting in an acute reduction of blood supply to a portion of the myocardium. As a result of lack of supply of oxygen and other nutrients to the myocardium, death occurs in some muscle cells of the heart.

Prevention and treatments of coronary heart disease (i.e., myocardial infarction and ischemic heart disease) include coronary artery bypass, angioplasty, stent placement, atherectomy, and combined therapies of drug treatment and surgery. In addition, the development of new therapeutic agents capable of limiting the extent of myocardial injury, i.e., the extent of myocardial infarction, is a major concern of modern cardiology.

Angiogenesis, the growth of new blood vessels, is a complex process involving disruption of vascular basement membranes, migration and proliferation of endothelial cells, and subsequent blood vessel formation and maturation. (See, e.g., Folkman et al., 1992, J Biol. Chem. 267: 10931 and Fan et al., 1995, Trends Pharmacol Sci. 16: 57-66). Angiogenesis can involve endothelial cell and pericyte activation; basal lamina degradation; migration and proliferation (i.e., cell division) of endothelial cells and pericytes; formation of a new capillary vessel lumen; appearance of pericytes around the new vessels; development of a new basal lamina; capillary loop formation; persistence of involution, differentiation of the new vessels; and, capillary network formation and, eventually, organization into larger microvessels. Several mediators, known as angiogenic growth factors are known to elicit angiogenic responses.

Increasing blood supply through the delivery of angiogenic growth factors may improve cardiac function following a myocardial infarction. For example, in the initial stage of an infarct, the core consists of necrotic tissue while the edges, or border zone, contain cardiomyocytes that are at risk. These at-risk myocytes will eventually become necrotic and produce a larger infarct if blood supply is not restored to them. Restoring blood supply to the infarct regions through delivery of angiogenic growth factors, a concept known as therapeutic angiogenesis, may salvage the at-risk cardiomyocytes and reduce infarct expansion with the ultimate goal of improving cardiac function and decreasing the morbidity and mortality associated with coronary heart disease.

Vascular endothelial growth factor (VEGF) is the most widely studied angiogenic growth factor. The VEGF gene and protein are known to be upregulated in ischemic myocardium. (See, e.g., Hashimoto, E. et al., 1994, Physiol 267: H1948-54; Heba, G. et al., 2001, J. Vasc. Res. 38:288-300). Although VEGF is known to induce angiogenesis, delivery of both plasmid VEGF and skeletal myoblasts genetically engineered to express VEGF have had deleterious effects. For example, constitutive expression of VEGF by high doses of retroviral transduced skeletal myoblasts in murine hearts induced the formation of intramural hemangiomas. (See, e.g., Lee, R. J. et al., 2000, Circulation 102:898-901). Similarly, injection of VEGF resulted in vascular tumors when injected into either the myocardium or hind limb muscle. (See, e.g., Schwarz E. R. et al., 2000, J. Am. Coll. Cardiol. 35:1323-1330; Isner, J. M. et al., 1996, Lancet 348:370-374). As studies have demonstrated deleterious effects associated with VEGF, its application towards therapeutic angiogenesis in cardiovascular tissue may be problematic.

A particular growth factor, pleiotrophin (PTN) is a 136 amino acid heparin-binding, secretory protein which has been shown to induce mitogenesis, angiogenesis, and neurite and glial process outgrowth in vitro. (See, e.g., Yeh, H. J., et al., 1998, J. Neurosci. 18, 3699-707; Courty, J. et al., 1991, Biochem. Biophys. Res. Commun. 180, 145-151; Fang, W. et al., 1992, J. Biol. Chem. 267, 25889-25897; Rauvala, H. et al., 1989, Biosci. Rep. 9, 1-12). PTN has been most widely studied in the brain and for its involvement in embryogenesis and perinatal growth. PTN mRNA is upregulated in macrophages, astrocytes, and endothelial cells in a focal cerebral ischemic rat model (See, Yeh, H. J., inter alia, supra).

Alternative approaches to the current methods of preventing and treating cardiovascular diseases are needed. Angiogenic growth factors such as PTN may provide a new means for treatment and therapy of cardiovascular diseases. As described by the present invention, the delivery and application of PTN to ischemic cardiac tissue results in stimulating or inducing blood vessel formation restoring blood supply to the cardiac tissue. Such a treatment may improve cardiac function and may decrease the morbidity and mortality associated with cardiovascular disease, and may provide an alternative approach to current methods of preventing and treating coronary heart disease.

### SUMMARY OF THE INVENTION

The present invention provides compositions comprising PTN (PTN compositions). In one aspect, PTN compositions induce or stimulate angiogenesis in vivo. In a particular aspect, PTN compositions induce or stimulate angiogenesis in cardiovascular tissue, and more particularly in cardiovascular tissue suffering from or at risk of suffering from ischemia. In a more specific aspect, PTN compositions induce or stimulate angiogenesis in cardiovascular tissue which is diseased or damaged as a result of conditions such as myocardial ischemia, atherosclerosis or myocardial infarction. PTN compositions of the present invention include vectors that produce and secrete biologically active PTN (PTN vectors). The PTN vectors of the present invention comprise a promoter operably linked to a nucleic acid sequence encoding a PTN protein. The PTN vectors of the present invention are capable of inducing angiogenesis in vivo.

The present invention also provides methods of inducing or stimulating angiogenesis in vivo. In one aspect, methods of the present invention comprise contacting the site with a PTN composition in an amount effective to induce or stimulate angiogenesis. In another aspect, the method comprises administering to a site or a subject in need, a PTN vector in an amount effective to induce or stimulate angiogenesis. In a particular aspect, the methods of the present induce or stimulate angiogenesis in cardiovascular tissue and more particularly, cardiovascular tissue suffering from or at risk of suffering from ischemia. In a specific aspect, the methods of the present invention induce or simulate angiogenesis in cardiovascular tissue which is diseased or damaged as a result of conditions such as myocardial ischemia, atherosclerosis or myocardial infarction.

The present invention also provides methods for enhancing the level of perfusion of blood to a target tissue. In a preferred aspect, the target tissue is ischemic tissue. The methods of the present invention comprise administering to the target tissue a PTN composition in an amount effective to induce or stimulate angiogenesis in the target tissue. In a particular aspect, the methods of the present invention comprise administering to the target tissue a PTN vector in an amount effective to induce or stimulate angiogenesis. In a preferred aspect, the target tissue suffers from or is at risk of suffering from ischemic damage. In a specific aspect, the target tissue is cardiovascular tissue which is diseased or damaged as a result of conditions such as myocardial ischemia, atherosclerosis or myocardial infarction.

The present invention further provides methods for treating a target tissue suffering from or at risk of suffering of ischemic damage. In a preferred aspect, the target tissue is cardiovascular tissue. The methods of the present invention comprise administering to the target tissue a PTN composition in an amount effective to induce or stimulate angiogenesis in the target tissue. In a particular aspect, the methods of the present invention comprise administering to the target tissue a PTN vector in an amount effective to induce or stimulate angiogenesis.

Also provided by the present invention are methods of preventing or reducing myocardial tissue damage resulting, for example, from ischemia. The methods of the present invention comprise administering to a subject in need an effective amount of a PTN composition. In one aspect, the method comprises administering to a subject in need a PTN vector in an amount effective to induce or stimulate angiogenesis in myocardial tissue. The method may be used to prevent myocardial tissue damage or protect cardiovascular tissue from damage during surgery. The methods of the present invention may also be used in subjects with ongoing cardiac (acute coronary syndromes, e.g. myocardial infarction or unstable angina) conditions.

The present invention also provides methods of preventing or treating cardiovascular diseases. The methods of the present invention are useful for preventing or treating diseases such as ischemia, myocardial infarction, and reperfusion injury. Yet another preferred aspect is a method of treating, preventing or inhibiting a cardiovascular disease in subject, such as angina pectoris, myocardial infarction, cardiovascular ischemia, and cardiovascular tissue damage. The methods of the invention are particularly useful for treating cardiovascular disorders including, for example, coronary artery disease, such as atherosclerosis, angina pectoris, myocardial infarction; myocardial ischemia and cardiac arrest, and cardiac bypass.

### BRIEF DESCRIPTION OF THE DRAWINGS

FIG. 1 shows time course of pleiotrophin (PTN) mRNA levels following ischemia in rat myocardium. PTN mRNA begins to elevate at day 3 and returns close to baseline by day 30.

FIG. 2A-2B show PTN expression and activity. FIG. 2A shows ELISA data of PTN secreted by 293 cells that were transfected with a pCMV-PTN plasmid (▪) or a pCMV-β-gal plasmid (□). Cells transfected with the PTN plasmid synthesized and secreted PTN while those transfected with the β-gal plasmid did not. FIG. 2B shows results from the low attachment, anchorage independent growth assay. The results demonstrate that secreted PTN was biologically active. Increased absorbance relates to an increase in proliferation. (▴) PTN isolated from the media of 293 cells transfected with PTN plasmid increased proliferation of SW13 cells in a dose dependent manner similar to basic fibroblast growth factor (Δ), which was used as a positive control. Mammalian cells transfected with the PTN plasmid were capable of producing and secreting biologically active PTN.

FIG. 3 shows infarct capillary density. The injection of PTN plasmid increased capillary density compared to injection of β-gal plasmid. Capillary density of normal, non-ischemic myocardium is provided as a reference.

FIG. 4A-4B shows PTN plasmid induced arteriogenesis. Anti-alpha smooth muscle actin stained arterioles five weeks after plasmid injection into ischemic myocardium. FIG. 4A shows β-gal plasmid injection. FIG. 4B shows arteriole formation as a result of PTN plasmid injection. Note the increase in arteriole density following PTN plasmid injection

FIG. 5 shows infarct arteriole density. As demonstrated in this figure, the injection of PTN plasmid increased arteriole density compared to injection of β-gal plasmid.

FIG. 6A-6B shows a localized increase in arterioles. FIG. 6A shows anti-smooth muscle actin labeled arterioles. FIG. 6B shows normal and infarcted myocardium visualized in the corresponding H&E labeled section. The increase in arterioles is localized to the infarct scar.

FIG. 7 shows a PTN nucleic acid sequence (SEQ ID NO:1).

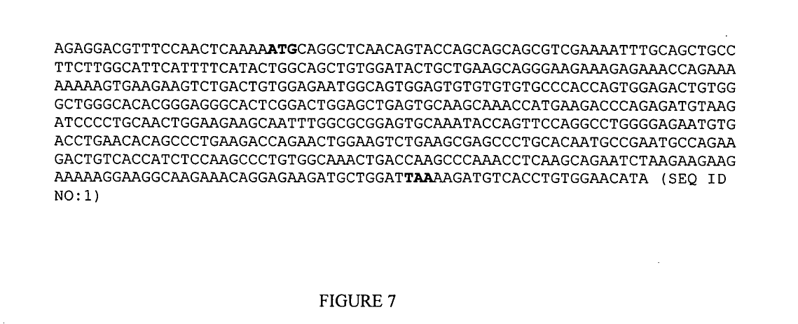

FIG. 8 shows that newly PTN-induced arterioles were functional. FIG. 8a shows newly induced arterioles revealed by anti-smooth muscle actin immunostaining of infarcted myocardium following administration of PTN. FIG. 8b shows microbead perfusion of the same section stained in 8a. FIG. 8c shows and overlay of 8a and 8b that demonstrates that new vessels formed by injection of PTN are functionally connected to the coronary vasculature.

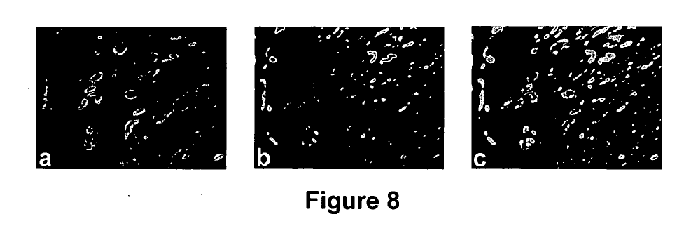

### DETAILED DESCRIPTION OF THE INVENTION

It is understood that this invention is not limited to the particular methodologies, protocols, cell lines, vectors, and reagents described, as these may vary. It is also to be understood that the terminology used herein is for the purpose of describing particular embodiments only, and is not intended to limit the scope of the present invention.

Unless defined otherwise, all technical and scientific terms used herein have the same meanings as commonly understood by one of ordinary skill in the art to which this invention belongs. The preferred methods, devices, and materials are now described, although any methods and materials similar or equivalent to those described herein can be used in the practice or testing of the present invention. All publications mentioned herein are incorporated by reference herein for the purpose of describing and disclosing, for example, cell lines, vectors, and methodologies reported in the publications which might be used in connection with the invention. Nothing herein is to be construed as an admission that the invention is not entitled to antedate such disclosure by virtue of prior invention. Each reference cited herein is incorporated by reference herein in its entirety.

### DEFINITIONS

As used herein, “pleiotrophin” or “PTN” refers to pleiotrophin protein obtained from any species, including human or other warm-blooded vertebrates, from any source, whether natural, synthetic, semi-synthetic or recombinant and includes protein fragments or peptides of pleiotrophin proteins.

As used herein, “pleiotrophin gene” or “pleiotrophin nucleic acid sequence” refers to any nucleic acid sequence encoding for a pleiotrophin protein obtained from any species, including human or other warm-blooded vertebrates, from any source, whether natural, synthetic, semi-synthetic or recombinant, and includes any nucleic acid sequence encoding active fragments of a pleiotrophin protein. A pleiotrophin gene or pleiotrophin nucleic acid sequence includes any nucleic acid sequence encoding active fragments of pleiotrophin, or any recombinant derivatives thereof.

As used herein “cardiovascular disease” or “cardiovascular disorder” refers to diseases of the heart and circulatory system and includes myocardial infarction, angina pectoris, myocardial ischemia, and related conditions as would be known by those of skill in the art which involve dysfunction of or tissue damage to the heart or vasculature. The term “cardiovascular disorders” refers to those disorders that can either cause ischemia or are caused by reperfusion of the heart. Examples include, but are not limited to, coronary artery disease, angina pectoris, myocardial infarction, cardiovascular tissue damage caused by cardiac arrest, cardiovascular tissue damage caused by cardiac bypass, cardiogenic shock, and related conditions that would be known by those of ordinary skill in the art or which involve dysfunction of or tissue damage to the heart or vasculature.

Production/Preparation of Pleiotrophin

Pleiotrophin may be isolated from natural sources or by recombinant production by methods well-known in the art. Nucleic acid sequences and amino acid sequences of PTN are described in the art. (See, e.g., Fang et al., 1992, J. Biol. Chem. 267:25889-25897; Li et al., 1990, Science 250:1690-1694; Lai et al., 1989, Biochem. Biophys. Res. Commun. 165:1096-1103; Kadomatsu et al., 1988, Biochem. Biophys. Res. Commun. 151:1312-1318; Tornornura et al., 1990, J Biol. Chem. 265:10765; Vrios et al., 1991, Biochem. Biophys. Res. Commun. 175:617-624; and Li et al., 1992, J Biol. Chem., 267:26011-26016. In a preferred embodiment, pleiotrophin is encoded by nucleic acid sequence set forth in SEQ ID NO 1, also described in PCT WO 96/02257. Other PTN sequences include the sequences disclosed in Zhang et al., 1999, J. Biol. Chem. 274:12959. Other PTN sequences include recombinant polypeptides comprising one or more regions of a full-length PTN.

PTN may be produced recombinantly using any of a variety of methods available in the art. (See, e.g., Sambrook et al., Molecular Cloning, A Laboratory Manual (2nd ed. 1989); Kriegler, Gene Transfer and Expression: A Laboratory Manual (1990); and Current Protocols in Molecular Biology Current Protocols in Molecular Biology (Ausubel et al., (eds.) 1997)).

Nucleic acid sequences encoding PTN can be typically cloned into intermediate vectors for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding PTN or production of protein. The nucleic acid encoding PTN can also be typically cloned into an expression vector, for administration to a plant cell, animal cell, preferably a mammalian cell or a human cell, fungal cell, bacterial cell, or protozoa cell.

To obtain expression of a cloned gene or nucleic acid, PTN can be typically subcloned into an expression vector that contains a promoter to direct transcription. Suitable bacterial and eukaryotic promoters are well known in the art. (See, e.g., Sambrook et al., Molecular Cloning, A Laboratory Manual (2nd ed. 1989); Kriegler, Gene Transfer and Expression: A Laboratory Manual (1990); and Current Protocols in Molecular Biology Current Protocols in Molecular Biology (Ausubel et al., (eds.) 1997). Bacterial expression systems for expressing PTN are available in, e.g., E. coli, Bacillus sp., and Salmonella. (See, e.g., Palva et al., 1983, Gene I (1983) 22: 229-235). Kits for such expression systems are commercially available. Eukaryotic expression systems for mammalian cells, yeast, and insect cells are well known in the art and are also commercially available.

The promoter used to direct expression of a PTN nucleic acid depends on the particular application. For example, a strong constitutive promoter can be typically used for expression and purification of the PTN. In contrast, when PTN is administered in vivo for gene regulation, either a constitutive or an inducible promoter can be used, depending on the particular use of the PTN. The promoter typically can also include elements that are responsive to transactivation, e.g., hypoxia response elements, Gal4 response elements, lac repressor response element, and small molecule control systems such as tet-regulated systems and the RU-486 system. (See, e.g., Gossen & Bujard, 1992, Proc. Natl. Acad. Sci. U.S.A. 89: 5547; Oligino et al., 1998, Gene Ther. 5: 491-496; Wang et al., 1997, Gene Ther. 4: 432-441; Neering et al., 1996, Blood 88: 1147-1155; and Rendahl et al., 1988, Nat. Biotechnol. 16: 757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding PTN, and may include signals for efficient polyadenylation of the transcript, transcriptional termination, ribosome binding sites, or translation termination. Additional elements of the cassette can include, e.g., enhancers, and heterologous spliced intronic signals.

The particular expression vector used to transport the genetic information into the cell can be selected with regard to the intended use of PTN, e.g., expression in plants, animals, bacteria, fungus, and protozoa. Standard bacterial expression vectors include plasmids such as pBR322 based plasmids, pSKF, pET23D, and commercially available fusion expression systems such as GST and LacZ. These fusion proteins can be used for purification of the PTN. Epitope tags can also be added to recombinant proteins to provide convenient methods of isolation, for monitoring expression, and for monitoring cellular and subcellular localization.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A.sup.+, pMTO10/A.sup.+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with an PTN encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques. (See, e.g., Colley et al., 1994, J. Biol. Chem. 264: 17619; Guide to Protein Purification, in Methods in Enzymology, Vol. 182 (Deutscher, ed., 1990). Transformation of eukaryotic and prokaryotic cells is performed according to standard techniques. (See, e.g., 1977, Morrison, J. Bact. 132: 349-351; Clark-Curtiss & Curtiss, Methods in Enzymology 101: 347-362 (Wu et al., eds. 1983)).

Alternatively, PTN may be obtained and purified from natural sources. The exact manner and protocol for purification of PTN from natural sources will depend on the source material and the particular PTN, as is well known in the art.

PTN may also be produced synthetically. For example, peptides, including peptide fragments of naturally occurring growth factors, with angiogenic activity, may be synthesized using solid phase techniques available in the art. Additionally, analogues, which act as growth factor mimics, may be synthesized using synthetic organic techniques available in the art, as described for example in: March, “Advanced Organic Chemistry”, John Wiley & Sons, New York, 1985. Analogues include small molecule peptide mimetics, as well as synthetic active peptides homologous to naturally occurring PTN or fragments thereof.

Treatments

Cardiovascular diseases which compositions of the present invention are useful for preventing or treating include but are not limited to arteriosclerosis; atherosclerosis; stroke; ischemia; endothelium dysfunctions, in particular those dysfunctions affecting blood vessel elasticity; peripheral vascular disease; coronary heart disease; myocardial infarction; cerebral infarction and restenosis.

Typically, cardiovascular tissue will be suffering from or be at risk of suffering from ischemic damage which results when the tissue is deprived of an adequate supply of oxygenated blood. There are many ways to determine if a tissue is at risk of suffering ischemic damage. Such methods are well known to physicians who treat such conditions. For example, in myocardial disease these methods include a variety of imaging techniques (e.g., radiotracer methodologies such as .sup.99m Tc-sestamibi scanning, x-ray, and MRI scanning) and physiological tests.

The ischemic damage may occur during organ transplantation or other surgical procedure. The compositions of the present invention may be administered prior to, during or shortly after, cardiac surgery or non-cardiac surgery. Particularly, the compositions of the present invention can be used as agents for myocardial protection before, during, or after coronary artery bypass grafting (CABG) surgeries, vascular surgeries, percutaneous transluminal coronary angioplasty (PTCA), organ transplantation, or nonrdiac surgeries. In addition, the compositions of the present invention may be used as agents for myocardial protection in patients presenting with ongoing cardiac (acute coronary syndromes, e.g. myocardial infarction or unstable angina) conditions.

Cardiovascular diseases such as atherosclerosis often require surgical procedures such as angioplasty. Angioplasty is often accompanied by the placement of a reinforcing a metallic tube-shaped structure known as a “stent” into a damaged coronary artery. For more serious conditions, open heart surgery such as coronary bypass surgery may be required. These surgical procedures entail using invasive surgical devices and/or implants, and are associated with a high risk of restenosis and thrombosis. Accordingly, the compositions of the present invention may be used as coatings on or delivered by surgical devices (e.g., catheters) and implants (e.g., stents).

PTN compositions can be administered alone or together with a known cardiovascular drug. Cardiovascular drugs for use in combination with the PTN compositions of the present invention to prevent or treat cardiovascular diseases include but are not limited to peripheral antiadrenergic drugs, centrally acting antihypertensive drugs (e.g., methyldopa, methyldopa HCI), antihypertensive direct vasodilators (e.g., diazoxide, hydralazine HCl), drugs affecting renin-angiotensin system, peripheral vasodilators, phentolamine, antianginal drugs, cardiac glycosides, inodilators (e.g., aminone, milrinone, enoximone, fenoximone, imazodan, sulmazole), antidysrhythmic drugs, calcium entry blockers, ranitine, bosentan, and rezulin.

Formulations and Routes of Administration

Carriers

PTN compositions of the present invention may be typically combined with a pharmaceutically acceptable carrier to form a pharmaceutical composition. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of the peptides or polypeptide complexes, or excipients or other stabilizers and/or buffers. Detergents can also used to stabilize or to increase or decrease the absorption of the pharmaceutical composition, see infra for exemplary detergents, including liposomal carriers. Pharmaceutically acceptable carriers and formulations for peptides and polypeptide are known to the skilled artisan and are described in detail in the scientific and patent literature. (See, e.g., Remington's, supra, and Banga, A. K., Therapeutic Peptides and Proteins. Formulation, Processing and Delivery Systems (1996) (Technomic Publishing AG, Basel, Switzerland).

Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. One skilled in the art would appreciate that the choice of a pharmaceutically acceptable carrier including a physiologically acceptable compound depends, for example, on the route of administration of the protein or polypeptide of the invention and on its particular physiochemical characteristics.

In a preferred aspect, PTN compositions are combined with carriers that are matrices in which PTN may be incorporated in a stable form while substantially maintaining its activity, and matrices which are biocompatible. Depending upon the selection of the delivery matrix, and the indication being treated, controlled release may be designed to occur on the order of hours, days, weeks, or longer.

The use of a controlled delivery matrix has many advantages. Controlled release permits dosages to be administered over time, with controlled release kinetics. In some instances, delivery of PTN needs to be continuous to the site where angiogenesis is needed, for example, over several weeks. Controlled release over time, for example, over several days or weeks, or longer, permits continuous delivery of PTN to obtain optimal angiogenesis in a therapeutic treatment. The controlled delivery matrix also is advantageous because it protects PTN from degradation in vivo in body fluids and tissue, for example, by proteases.

Controlled release from the delivery matrix may designed, based on factors such as choice of carrier, to occur over time, for example, for greater than about 12 or 24 hours. The time of release may be selected, for example, to occur over a time period of about 12 hours to 24 hours; about 12 hours to 42 hours; or, e.g., about 12 to 72 hours. In another embodiment, release may occur for example on the order of about 2 to 90 days, for example, about 3 to 60 days. In one embodiment, PTN is delivered locally over a time period of about 7-21 days, or about 3 to 10 days. PTN may be administered over 1, 2, 3 or more weeks in a controlled dosage. The controlled release time may be selected based on the condition treated. For example, longer times may be more effective for wound healing, whereas shorter delivery times may be more useful some cardiovascular applications.

Controlled release of the PTN, from the matrix in vivo may occur, for example, in the amount of about 1 ng to about 1 mg/day, for example, about 50 ng to about 500 μg/day, or, in one embodiment, about 100 ng/day. Delivery systems comprising the PTN and the carrier may be formulated that include, for example, 10 ng to 1 mg PTN, or in another embodiment, about 1 μg to about 500 μg, or, for example, about 10 μg to about 100 μg, depending on the therapeutic application.

The delivery matrix may be, for example, a diffusion controlled matrix system or an erodible system, as described, for example, in Lee, “Diffusion Controlled Matrix Systems”, pp. 155-198 and Ron and Langer, “Erodible Systems”, pp. 199-224, in “Treatise on Controlled Drug Delivery”, A. Kydonieus Ed., Marcel Dekker, Inc., New York 1992. The matrix may be, for example, a biodegradable material that can degrade spontaneously in situ and in vivo for example, by hydrolysis or enzymatic cleavage, e.g., by proteases. Optionally, a conjugate of the PTN and a polymeric carrier may be used.

The delivery matrix may be, for example, a naturally occurring or synthetic polymer or copolymer, for example in the form of a hydrogel. Exemplary polymers with cleavable linkages include polyesters, polyorthoesters, polyanhydrides, polysaccharides, poly(phosphoesters), polyamides, polyurethanes, poly(imidocarbonates) and poly(phosphazenes).

Polyesters include poly(a-hydroxyacids) such as poly(lactic acid) and poly(glycolic acid) and copolymers thereof, as well as poly(caprolactone) polymers and copolymers. In a preferred embodiment the controlled release matrix is a poly-lactide-co-glycolide. Controlled release using poly(lactide) and poly(glycolide) copolymers is described in Lewis, “Controlled Release of Bioactive Agents from Lactide/Glycolide Polymers” in “Biodegradable Polymers as Drug Delivery Systems”, Chasin and Langer, eds., Marcel Dekker, New York, 1990, pp. 1-41. Poly-lactide-coglycolides may be obtained or formed in various polymer and copolymer ratios, for example, 100% D,L-lactide; 85:15 D,L-lactide:glycolide; 50:50 D,Llactide:glycolide; and 100% glycolide, as described, for example, in Lambert and Peck, J. Controlled Release, 33:189-195 (1995); and Shively et al., J. Controlled Release, 33:237-243 (1995). The polymers can be processed by methods such as melt extrusion, injection molding, solvent casting or solvent evaporation.

The use of polyanhydrides as a controlled release matrix, and the formation of microspheres by hot-melt and solvent removal techniques is described in Chasin et al., “Polyanhydrides as Drug Delivery Systems,” in “Biodegradable Polymers as Drug Delivery Systems”, Chasin and Langer, Eds., Marcel Dekker, New York, 1990, pp. 42-70.

A variety of polyphosphazenes may be used which are available in the art, as described, for example in: Allcock, H. R., “Polyphosphazenes as New Biomedical and Bioactive Materials,” in “Biodegradable Polymers as Drug Delivery Systems”, Chasin and Langer, eds., Marcel Dekker, New York, 1990, pp. 163-193.

Polyamides, such as, poly(amino acids) may be used. In one embodiment, the polymer may be a poly(amino acid) block copolymer. For example, fibrinelastin and fibrin-collagen polymers, as well as other proteinaceous polymers, including chitin, alginate and gelatin may be used. In one embodiment, a silk elastin poly(amino acid) block copolymer may be used. Genetic and protein engineering techniques have been developed which pen-nit the design of silk elastin poly(amino acid) block copolymers with controlled chemical and physical properties. These protein polymers can be designed with silk-like crystalline amino acid sequence blocks and elastin-like flexible amino acid sequence blocks.

Poly(phosphoesters) may be used as the controlled delivery matrix. Poly(phosphoesters) with different side chains and methods for making and processing them are described in, for example, Kadiyala et al., “Poly(phosphoesters): Synthesis, Physiochemical Characterization and Biological Response,” in “Biomedical Applications of Synthetic Biodegradable Polymers”, J. Hollinger, Ed., CRC Press, Boca Raton, 1995, pp. 33-57. Polyurethane materials may be used, including, for example, polyurethane amide segmented block copolymers, which are described, for example, in U.S. Pat. No. 5,100,992. Poloxamer polymers may be used, which are polyoxyalkylene block copolymers, such as ethylene oxide propylene oxide block copolymers, for example, the Pluronic gels.

In another embodiment the controlled delivery matrix may be a liposome. Amphiphilic molecules such as lipid containing molecules may be used to form liposomes, as described in Lasic, “Liposomes in Gene Delivery,” CRC Press, New York, 1994, pp. 674. Exemplary lipids include lecithins, sphingomyelins, and phosphatidylethanolamines, phosphatidylserines, phosphatidylglycerols and phosphatidylinositols. Natural or synthetic lipids may be used. For example, the synthetic lipid molecule's used to form the liposomes may include lipid chains such as dimyristoyl, dipalmitoyl, distearoyl, dioleoyl and palmitoyl-oleoyl chains. Cholesterol may be included. Liposomes and methods for their formation also are described in Nassander, “Liposomes” in “Biodegradable Polymers as Drug Delivery Systems”, Chasin and Langer, Eds., Marcel Dekker, New York, 1990, pp. 261-338.

Collagen, albumin, and fibrinogen containing materials may be used, as described, for example, in Bogdansky, “Natural Polymers as Drug Delivery Systems”, in “Biodegradable Polymers as Drug Delivery Systems”, Chasin and Langer, Eds., Marcel Dekker, New York, 1990, pp. 231-259; Karen L. Christman^{a}, Qizhi Fang^{b}, Michael S. Yee^{c}, Kandice R. Johnson^{d}, Richard E. Sievers^{c}, Randall J. Lee^{a}, Enhanced Neovasculature Formation in Ischemic Myocardium Following Delivery of Pleiotrophin Plasmid in a Biopolymer. Biomaterials, 2004 (in press).

Drug delivery systems based on hyalurans, for example, including hyaluronan or hyaluronan copolymerized with a hydrophilic polymer or hylan, may be used, as described in U.S. Pat. No. 5,128,326.

Hydrogel materials available in the art may be used. Exemplary materials include poly(hydroxyethyl methacrylate) (poly(HEMA)), water-insoluble polyacrylates, and agarose, polyamino acids such as alginate and poly(L-lysine), poly(ethylene oxide) (PEO) containing polymers, and polyethylene glycol (PEG) diacrylates. Other examples of hydrogels include crosslinked polymeric chains of methoxy poly(ethylene glycol) monomethacrylate having variable lengths of the polyoxyethylene side chains, as described in Nagaoka, et al., in Polymers as Biornaterials (Shalaby, S. W., et al., Eds.), Plenum Press, 1983, p. 381. Hydrogels may be used that include hydrophilic and hydrophobic polymeric components in block (as disclosed in Okano, et al., 1981, J. Biomed. Mat. Research, 15, 393), or graft copolymeric structures (as disclosed in Onishi, et al., in Contemporary Topics in Polymer Science, (W. J. Bailey & T. Tsuruta, eds.), Plenum Publ. Co., New York, 1984, p. 149), and blends (as disclosed in Shah, Polymer, 28, 1212,1987; and U.S. Pat. No. 4,369,229).

Hydrogels comprising acrylic-terminated, water-soluble chains of polyether dl-polylactide block copolymers may be used. Hydrogels may comprise polyethylene glycol, a poly(ct-hydroxy acid), such as poly(glycolic acid), poly(DL-lactic acid) or poly(L-lactic acid) and copolymers thereof, or poly(caprolactone) or copolymers thereof. In one embodiment, the hydrogel may comprise a copolymer of poly(lactic acid) and poly(glycolic acid), also referred to herein as a poly-lactide-co-glycolide (PLGA) polymer. Hydrogels may be used that are polymerized and crosslinked macromers, wherein the macromers comprise hydrophilic olig9mers having biodegradable monomeric or oligomeric extensions, terminated on the free ends thereof with end cap monomers or oligomers capable of polymerization and cross linking. The hydrophilic core itself may be degradable, thus combining the core and extension functions. The macromers are polymerized for example using free radical initiators under the influence of long wavelength ultraviolet light, visible light excitation or thermal energy. Biodegradation occurs at the linkages within the extension oligomers and results in fragments which are non-toxic and easily removed from the body.

Additionally, the delivery matrix may include a targeting ligand which permits targeted delivery of the PTN to a preselected site in the body. The targeting ligand is a specific binding moiety which is capable of binding specifically to a site in the body. For example, the targeting ligand may be an antibody or fragment thereof, a receptor ligand, or adhesion molecule selective or specific to the desired target site. Examples of target sites include vascular intercellular adhesion molecules (ICAMs), and endothelial cell-surface receptors, such as WP3. Embodiments of delivery matrices including a targeting ligand include antibody-conjugated liposomes, wherein the antibody is linked to the liposome via an avidin-biotin linker, which are described, for example, in Sipkins, 1995, Radiology, 197:276 (Abstract); and Sipkins et al., 1995, Radiology 197:129 (1995) (Abstract).

Formulations and Administration

PTN compositions, optionally in a carrier, or formulation thereof, may be administered by a variety of routes known in the art including topical, oral, parenteral (including intravenous, intraperitoneal, intramuscular and subcutaneous injection as well as intranasal or inhalation administration) and implantation. The delivery may be systemic, regional, or local. Additionally, the delivery may be intrathecal, e.g., for CNS delivery. For example, administration of the PTN for the treatment of wounds may be by topical application of the PTN to the wound, systemic administration by enteral or parenteral routes, or local or regional injection or implantation. The PTN may be formulated into appropriate forms for different routes of administration as described in the art, for example, in “Remington: The Science and Practice of Pharmacy”, Mack Publishing Company, Pennsylvania, 1995.

PTN, optionally incorporated in a controlled release matrix, may be provided in a variety of formulations including solutions, emulsions, suspensions, powders, tablets and gels. The formulations may include excipients available in the art, such as diluents, solvents, buffers, solubilizers, suspending agents, viscosity controlling agents, binders, lubricants, surfactants, preservatives and stabilizers. The formulations may include bulking agents, chelating agents, and antioxidants. Where parenteral formulations are used, the formulation may additionally or alternately include sugars, amino acids, or electrolytes. Excipients include polyols, for example of a molecular weight less than about 70,000 kD, such as trehalose, mannitol, and polyethylene glycol. See for example, U.S. Pat. No. 5,589,167. Exemplary surfactants include nonionic surfactants, such as Tweene surfactants, polysorbates, such as polysorbate 20 or 80, etc., and the poloxamers, such as poloxamer 184 or 188, Pluronic(r) polyols, and other ethylene/polypropylene block polymers, etc. Buffers include Tris, citrate, succinate, acetate, or histidine buffers. Preservatives include phenol, benzyl alcohol, metacresol, methyl paraben, propyl paraben, benzalconium. chloride, and benzethonium chloride. Other additives include carboxymethylcellulose, 15 dextran, and gelatin. Stabilizing agents include heparin, pentosan polysulfate and other heparinoids, and divalent cations such as magnesium and zinc.

PTN, optionally in combination with a controlled delivery matrix, may be processed into a variety of forms including microspheres, microcapsules, microparticles, films, and coatings. Methods available in the art for processing drugs into polymeric carriers may be used such as spray drying, precipitation, and crystallization. Other methods include molding techniques including solvent casting, compression molding, hot-melt microencapsulation, and solvent removal microencapsulation, as described, for example in Laurencin et al., “Poly(anhydrides)” in “Biomedical Applications of Synthetic Biodegradable Polymers”, J. Hollinger, Ed., CRC Press, Boca Raton, 1995, pp. 59-102.

In one embodiment, it is advantageous to deliver PTN locally in a controlled release carrier, such that the location and time of delivery are controlled. Local delivery can be, for example, to selected sites of tissue, such as a wound or other area in need of treatment, or an area of inadequate blood flow (ischemia) in tissue, such. as, ischemia heart tissue or other muscle such as peripheral.

PTN, optionally in combination with a carrier, such as a controlled release matrix, also may be administered locally near existing vasculature in proximity to an ischemic area for an indication such as an occlusive vascular disease, to promote angiogenesis near the area being treated.

Delivery of PTN Vector

The present invention also involves the administration of a vector comprising a nucleic acid sequence encoding PTN in a localized manner to the target tissue. The target tissue may be any ischemic tissue. In a preferred embodiment the target tissue is cardiovascular tissue. While any suitable means of administering the vector to the target tissue can be used within the context of the present invention, a preferred embodiment such as localized administration to the target tissue is accomplished by directly injecting the vector into the target tissue or by topically applying the vector to the target tissue. Any suitable injection device can be used within the context of the present invention. Such injection devices include, but are not limited to, that described in U.S. Pat. No. 5,846,225, which is directed to a gene transfer delivery device capable of delivering simultaneous multiple injections. Another example of an injection device which can be used within the context of the present invention includes minimally invasive injection devices. Such devices are capable of accessing the heart, for example, through small incisions of less than 5 inches and are designed to provide injections through a single lumen, in contrast to the multiple injection device described above. To allow for the need for multiple injections with a specific geometry, a marking system can be employed so that the sites of previous injections are well delineated. Minimally invasive injection devices can comprise injector tips which are flexible and steerable to allow access via small incisions to the curved outer surface of the heart, for example, which exists at varying angles with respect to the limited aperture window required with minimally invasive surgeries.

Furthermore, the PTN vector can be administered to any suitable surface, either internal or external, of the target tissue. For example, with respect to directly injecting the PTN vector into cardiac tissue, it is contemplated that such an injection can be administered from any suitable surface of the heart (i.e., endocardially and/or epicardially).

While administration of a dose of the PTN vector can be accomplished through a single application (e.g., a single injection or a single topical application) to the target tissue, preferably, administration of the dose is via multiple applications of the PTN vector. The multiple applications can be 2, 3, 4, 5, or more applications, preferably 5 or more applications, more preferably 8 or more applications, and most preferably at least 10 (e.g., 10, 15, or 20) applications. Multiple applications provide an advantage over single applications in that they can be manipulated by such parameters as a specific geometry defined by the location on the target tissue where each application is administered. The administration of a single dose of the PTN vector via multiple applications can be better controlled, and the effectiveness with which any given dose is administered can be maximized.

The specific geometry of the multiple applications may be defined by the location on the target tissue, either in two- or three-dimensional space, where each application of the PTN vector is administered. The multiple applications preferably are spaced such that the points of application are separated by up to about 4 cm (e.g., about 0.5-4 cm), more preferably up to about 3 cm (e.g., about 1-3 cm), and most preferably up to about 2 cm (e.g., about 1-2 cm). With respect to the specific geometry of the multiple applications in two-dimensional space, the specific geometry is defined by a plane (i.e., a cross-section of the target tissue) in which the multiple applications lie. The plane defined by the multiple applications can lie at a constant distance from the surface of the target tissue (i.e., substantially parallel to the surface of the target tissue), the depth of the plane, or, alternatively, the plane can lie at an angle with respect to the surface of the target tissue. Preferably, a single application will be administered for about every 0.5-15 cm.sup.2 of the plane, more preferably for about every 1-12 cm.sup.2 of the plane, and most preferably for about every 1.5-7 cm of the plane. The depth of the plane is preferably about 1-10 mm, more preferably about 2-7 mm, and most preferably about 3-5 mm. In three-dimensional space, a single application preferably is administered for up to about 50 cm.sup.3 (e.g., about 0.5-50 cm.sup.3) of target tissue, more preferably for up to about 35 cm.sup.3 (e.g., about 1-35 cm.sup.3) of target tissue, and most preferably for up to about 15 cm.sup.3 (e.g., about 3-15 cm.sup.3) of target tissue. Furthermore, the multiple applications can define any suitable pattern or specific geometry. Therefore, for example, in two-dimensional space, the multiple applications can define a square whereas in three-dimensional space the multiple applications can define a cube.

Another parameter of the multiple applications which can be manipulated is the time differential between each application. Preferably, each of the multiple applications is administered within about 10 minutes (e.g., about 0.5-10 minutes) of each other, more preferably within about 8 minutes (e.g., about 0.5-8 minutes) of each other, and even more preferably within about 6 minutes (e.g., about 1-6 minutes) of each other. Most preferably, all of the multiple applications of the single dose are administered within the aforesaid time frames. Optimally, each of the multiple applications is administered substantially simultaneously.

When administering the PTN vector to a target tissue which is affected by or at risk of being affected by a vascular occlusion, it is desirable that the administration is such that the PTN vector is able to contact a region reasonably adjacent to the source and the terminus for the collateral blood vessel formation, as well as the area there between, which will function as a bypass to the vascular occlusion. It is not believed to be necessary to have the PTN vector actually contact the precise sites of the source and the terminus for the collateral blood vessel formation. However, within the context of multiple applications of the VTN vector, it may be desirable that the specific geometry of the multiple applications be defined to allow the PTN vector to contact or reach a region including the source, the terminus, and the area there between for the collateral blood vessel formation, preferably to actually contact the precise sites of the source and the terminus for the collateral blood vessel formation, along with the area there between.

In addition, local delivery of PTN at the site of angioplasty, stent placement, or atherectomy may provide the means to precisely target the cells of interest and, thereby, achieve a higher concentration of the gene within the arterial wall than can be obtained by systemic administration. Furthermore, local catheter-based gene delivery would safeguard against systemic toxicity.

Furthermore, it is contemplated by the present invention that administration of the PTN vector to the target tissue can be accomplished either in vivo or ex vivo. Therefore, for example, the target tissue can be removed from the recipient of the present inventive method, can be treated with a PTN substance, and then can be reimplanted into the recipient.

PTN Vector

A number of vectors (viral or non-viral) known in the art may be used to achieve PTN protein expression in cardiovascular relevant sites such as myocardium, vascular endothelium, and skeletal muscle.

Any nucleic acid sequence encoding a PTN peptide and operably linked to suitable expression signals can be used within the context of the present invention. Whereas, the nucleic acid sequence can be operably linked to any suitable set of expression signals, preferably, the expression of the DNA is under the control of the cytomegalovirus (CMV) immediate early promoter.

Viral vectors, such as retroviruses, adenoviruses, adenoassociated viruses and herpes viruses, are often made up of two components, a modified viral genome and a coat structure surrounding it. (See, e.g., Smith et al., 1995, Ann. Rev. Microbiol. 49:807-838). Alternatively, the vectors may be introduced in naked form or coated with proteins other than viral proteins. Most current vectors have coat structures similar to a wild-type virus. This structure packages and protects the viral nucleic acid and provides the means to bind and enter target cells. However, the viral nucleic acid in a vector designed for gene therapy is changed in many ways. The goals of these changes are to disable growth of the virus in target cells while maintaining its ability to grow in vector form in available packaging or helper cells, to provide space within the viral genome for insertion of exogenous DNA sequences, and to incorporate new sequences that encode and enable appropriate expression of the gene of interest. Thus, vector nucleic acids generally comprise two components: essential cis-acting viral sequences for replication and packaging in a helper line and the transcription unit for the exogenous gene. Other viral functions are expressed in trans in a specific packaging or helper cell line. Methods of making viral vectors comprising nucleic acid sequences encoding angiogenic factors are known in the art and may be applied to the present invention. (See, e.g, U.S. Patent Application No. 20020019350).

Nonviral nucleic acid vectors include, for example, plasmids, RNAs, antisense oligonucleotides (e.g., methylphosphonate or phosphorothiolate), polyamide nucleic acids, and yeast artificial chromosomes (YACs). Such vectors typically include an expression cassette for expressing a protein or RNA. The promoter in such an expression cassette can be constitutive, cell type-specific, stage-specific, and/or modulatable (e.g., by hormones such as glucocorticoids; MMTV promoter). Transcription can be increased by inserting an enhancer sequence into the vector. Enhancers are cis-acting sequences of between 10 to 300 bp that increase transcription by a promoter. Enhancers can effectively increase transcription when either 5′ or 3′ to the transcription unit. They are also effective if located within an intron or within the coding sequence itself. Typically, viral enhancers are used, including SV40 enhancers, cytomegalovirus enhancers, polyoma enhancers, and adenovirus enhancers. Enhancer sequences from mammalian systems are also commonly used, such as the mouse immunoglobulin heavy chain enhancer.

Vectors of the present invention can also include a selectable marker gene. Examples of suitable markers include, the dihydrofolate reductase gene (DHFR), the thymidine kinase gene (TK), or prokaryotic genes conferring drug resistance, gpt (xanthine-guanine phosphoribosyltransferase, which can be selected for with mycophenolic acid; neo (neomycin phosphotransferase), which can be selected for with G418, hygromycin, or puromycin; and DHFR (dihydrofolate reductase).

Nonviral vectors encoding products can be introduced into an animal by means such as lipofection, biolistics, virosomes, liposomes, immunoliposomes, polycation: nucleic acid conjugates, naked DNA, artificial virions, agent-enhanced uptake of DNA, ex vivo transduction. Lipofection is described in the art. (See, e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides. (See, e.g., WO 91/17424, WO 91/16024). In a preferred embodiment, the PTN vector of the present invention is incorporated by way of naked DNA. Naked DNA or lipofection complexes can be used to transfer large (e.g., 50-5,000 kb) exogenous polynucleotides into cells. This property of nonviral vectors is particularly advantageous since many genes which can be delivered by therapy span over 100 kilobases (e.g., amyloid precursor protein (APP) gene, Huntington's chorea gene).

The technique using direct DNA injection into myocardium has several advantages compared with other previously described methods of gene therapy. First, infectious viral vectors are not required, eliminating the possibility of persistent infection of the host. Second, a previous study (Wolff J A, Malone R W, Williams P, Chong W, Acsadi G, Jani A, Felgner P L: Direct gene transfer into mouse muscle in vivo. Science 1990; 247:1465-1468) has suggested that recombinant DNA taken up and expressed in skeletal myocytes persists as an episome and therefore does not have the same potential for host cell mutagenesis as do retroviral vectors that integrate into the host chromosome. Finally, this method does not require the growth of recipient cells in vitro.

Direct injection into the myocardium is useful for the treatment of many acquired and inherited cardiovascular diseases in particular, by stimulating collateral circulation in areas of chronic myocardial ischemia by expressing recombinant angiogenesis factors locally in the ventricular wall.

Pharmaceutical Composition and Dosage

The PTN vector may be administered to the target tissue in a pharmaceutical composition which comprises a pharmaceutically acceptable carrier and the PTN vector. Any suitable pharmaceutically acceptable carrier can be used within the context of the present invention, and such carriers are well known in the art. The choice of carrier will be determined, in part, by the particular site to which the composition is to be administered and the particular method used to administer the composition. Formulations suitable for injection include aqueous and non-aqueous solutions, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous injection solutions and suspensions can be prepared from sterile powders, granules, and tablets of the kind previously described. Preferably, the pharmaceutically acceptable carrier is a buffered saline solution.

Although any suitable volume of carrier can be utilized within the context of the present invention, it may be desirable that the PTN vector be administered in small volumes of carrier so that the tissue to be vascularized (i.e., the target tissue) is perfused with the PTN vector but the angiogenic vector is not carried by the blood, lymphatic drainage, or physical mechanisms (e.g., gravitational flow or osmotic flow) to tissues which have not been targeted.

The determination of the proper dosage of the PTN vector can be easily made by those of ordinary skill in the art. However, generally, certain factors will impact the dosage which is administered.

Although the proper dosage is such that angiogenesis is induced in the target tissue, preferably, the dosage is sufficient to have a therapeutic and/or prophylactic effect on target tissue which is affected by or at risk of being affected by a vascular occlusion which may lead to ischemic damage of the tissue. Additionally, the dosage should be such that induction of angiogenesis in non-targeted tissue is minimized.

Therapeutic Kits

Kits can be supplied for therapeutic or diagnostic uses. Kits of the present invention comprise PTN compositions. In one embodiment the pharmaceutical formulation of the invention is in a lyophilized form, which can be placed in a container. The complexes, which can also be conjugated to a label, or unconjugated, are included in the kits with buffers, such as Tris, phosphate, carbonate, stabilizers, biocides, inert proteins, e.g., serum albumin, or the like, and a set of instructions for use. Generally, these materials will be present in less than about 5% wt. based on the amount of complex and usually present in total amount of at least about 0.001% wt. based again on the protein concentration. Frequently, it will be desirable to include an inert extender or excipient to dilute the active ingredients, where the excipient can be present in from about 1% to 99% wt. of the total composition.

Other features of the invention will become apparent in the course of the following examples which are given for illustration of the invention and are not intended to be limiting.

### EXAMPLES

### Example 1

### Induction of Myocardial Ischemia

To induce myocardial ischemia, female Sprague-Dawley Rats (225-250 g) were anesthetized with ketamine (90 mg/kg) and xylazine (10 mg/kg). A single stitch of 7-0 Ticron suture was placed under the left anterior descending (LAD) coronary artery. The suture was tightened to completely occlude the artery. A set of control animals underwent sham surgeries where the suture was not tightened. The animals were sacrificed either 30 min, 24 hr, 1 day, 3 days, 7 days, 14 days, or 30 days after placement of the suture (n=3 per group). The hearts were rapidly excised and the ischemic portion of the left ventricle (LV) or corresponding area of the controls was separated and flash frozen in liquid nitrogen.

### Example 2

### RNA Isolation and RT-PCR

RNA from each sample was isolated using a Mini Rneasy Kit (Qiagen, Valencia, Calif.). RT-PCR was performed using 0.5 μg RNA from each sample, Qiagen's One-Step RT-PCR kit, and PTN sense (5′-CTGTGGAGAATGGCAATGGA-3′) and antisense (5′-CGGCATTGTGCAGAGCTCT-3′) primers. The PTN primers mapped nucleotides 406 to 656 of the rat PTN sequence. 22 cycles were run under the following conditions: 94° C. for 1 min, 60° C. for 1 min, 72° C. for 1 min, and a final extension of 72° C. for 10 min. The RT-PCR products were electrophoresed in a 2% agarose gel containing 0.005% ethidium bromide.

### Example 3

### PTN Expression Constructs

The 580 base pair human PTN open reading frame (ORF) was isolated by RT-PCR from a human adenocarcinoma cell line (SW13), and was subcloned into the HindIII and XbaI sites of pRc/CMV2 (Invitrogen, Carlsbad, Calif.) to generate pRC/CMV2-PTN. The CMV promoter/enhancer and PTN open reading frame were shuttled from the pRC/CMV2-PTN to pIRES (BD Biosciences Clontech, Palo Alto, Calif.) to generate pCMV-PTN-IRES. The β-gal ORF from CMVβ (BD Biosciences Clontech) was shuttled into pCMV-PTN-IRES to generate pCMV-PTN-IRES-β-gal-neo. The control plasmid for cell culture was pCMV-IRES-β-gal-neo, which is similar to the pCMV-PTN-IRES-β-gal-neo but does not contain the PTN open reading frame. The control plasmid for injections was a pCMV-β-gal plasmid (Invitrogen) which is a similar construct without the PTN gene.

### Example 4

### ELISA

Human embryonic kidney cells (293 cells) were transfected by calcium phosphate precipitation with pCMV-PTN-IRES-β-gal-neo or pCMV-IRES-β-gal-neo control plasmid. Stably expressing cell lines were selected using geneticin (G418, Gibco Invitrogen). Following selection cells were grown to confluency and an aliquot of cell culture medium was taken for ELISA analysis. Media and a PTN standard (R&D Systems, Minneapolis, Minn.) were serially diluted (in triplicate and duplicate, respectively) in coating buffer (50 mM Na₂CO₃, pH 8.9) in a 96 well plate and incubated at RT for one hour. Wells were blocked with blocking buffer (1% BSA, 0.05% Tween in PBS) for one hour at RT. Wells were washed and a horse radish peroxidase-conjugated anti-human PTN antibody (900 ng/ml; R&D Systems) was added and incubated for 1 hour at RT. Plates were washed and 100 ul of TMB substrate was added to each well and incubated for 10 minutes. 50 ul of 1% H₂SO₄ was added and the plate was read at 450 nm.

### Example 5

### Low Attachment, Anchorage Independent Growth Assay

A 96 well plate was coated with poly 2-hydroxyethyl methacrylate (Sigma, St. Louis, Mo.), which prevents cell adherence. Growth factor (either PTN or bFGF) was serially diluted in triplicate in media (1% FBS/IMDM) and 2000 serum-starved SW13 cells (epithelial adenocarcinoma) were placed in each well. The plate was incubated for three days at which time MTS (Promega, Madison, Wis.), a tetrazolium compound that is reduced by viable cells into a formazan product, was added to the wells. The amount of formazan product, which directly correlates with the amount of proliferation, was measured by reading absorbance at 490 nm with a microplate reader (BioTek ELX 800).

### Example 6

### Plasmid Injections

Ischemia reperfusion model was used. (Sievers et al., 1989, Magn. Reson. Med. 10: 172-181). Rats were anesthetized, their chests were opened, and a suture was placed under the LAD as described above. The suture was tightened to occlude the LAD for 17 minutes and then removed to allow for reperfusion. Ten minutes after occlusion, 250 μg of either a control pCMV-β-gal plasmid or a pCMV-PTN plasmid in 50 μl saline was injected into the ischemic LV through a 30-gauge needle. The chest was then closed and the animals were allowed to recover. The technique used results in an acute infarct size of approximately 30% of the LV with reperfusion.

### Example 7

### Histology

The rats (n=6 per group) were euthanized with a pentobarbital overdose (200 mg/kg) five weeks after infarction at which point the remodeling process in the rat is complete. Two additional animals injected with PTN plasmid were sacrificed after 3 months. The hearts were rapidly excised and fresh frozen in Tissue Tek O.C.T. freezing medium (Sakura, Torrance, Calif.). They were then sectioned into 10 μm slices and stained with H&E. Five slides, equally distributed through the infarct area were taken from each heart as a representative sample and stained with an anti-smooth muscle actin antibody (1:75 dilution; Dako, Carpinteria, Calif.) to label arterioles. In order to visualize labeled arterioles, sections were incubated with a Cy-3 conjugated anti-mouse secondary antibody (1:100 dilution; Sigma). Arterioles in each section were quantified using the following criteria: 1) positive for smooth muscle labeling, 2) within the infarct scar, 3) having a visible lumen and 4) a diameter ≧10 μm. The scar area was measured using SPOT 3.5.1 software (Diagnostic Instruments, Sterling Heights, Mich.) and arteriole densities were calculated. 5 additional slides were taken from each heart and stained for capillaries. Capillaries were labeled with a biotinylated Griffonia simplicifolia lectin (GS-1; Vector Labs, Burlingame, Calif.) and visualized using the LSAB2 System (Dako) as previously described procedure (Keller et al., 2001, Circulation 104:2063-2068). The capillaries in five high magnification fields within the infarct area of each slide were counted and vessel density was calculated.

### Example 8

### Upregulation of PTN Gene Expression in Response to Ischemia

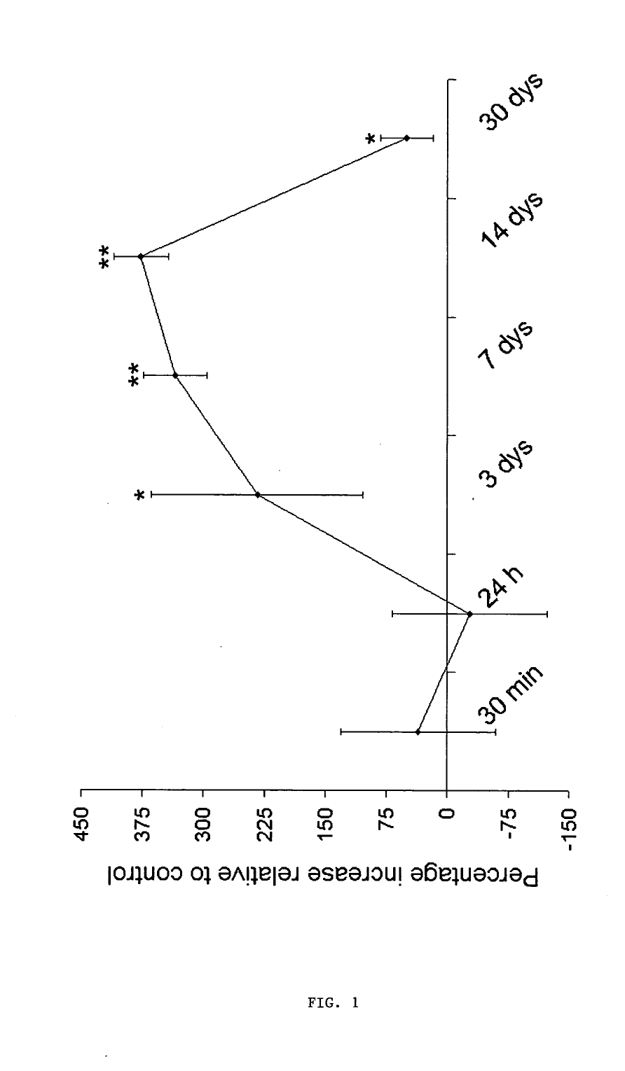

To begin to determine the role of pleiotrophin as an in vivo angiogenic agent for the myocardium, the expression of the PTN gene following ischemia in rat myocardium by reverse transcriptase-polymerase chain reaction (RT-PCR) was analyzed. Compared to sham operated controls, PTN mRNA becomes significantly elevated at 3 days following ischemia and returns close to the baseline level after 30 days (FIG. 1). PTN mRNA levels did not significantly change at 30 minutes and 24 hours following ischemia. At 3 days, 7 days, and 14 days PTN mRNA was elevated approximately 230%, 330%, and 380% respectively compared to non-ischemic myocardium. The increased expression of PTN in response to ischemia demonstrates that PTN is involved in angiogenesis.

### Example 9

### Expression of PTN

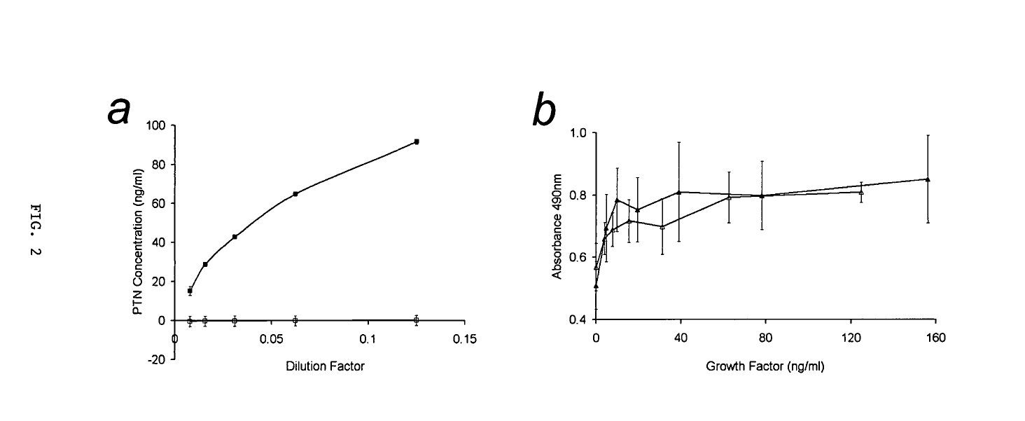

A pCMV plasmid encoding the pleiotrophin gene was constructed and transfected into 293 human embryonic kidney cells with either this plasmid or a pCMV-β-galactosidase (β-gal) control plasmid. An ELISA assay, was used to determine whether the mammalian cells transfected with the pCMV-PTN plasmid were capable of producing and secreting PTN. PTN is secreted by cells transfected with the PTN plasmid, but as shown in FIG. 2A, cells transfected with β-gal plasmid do not secrete PTN. The decreasing slope seen in the lower dilutions of medium from PTN plasmid transfected cells is due to factors present in the media that interfere with the ELISA (data not shown). The aliquot of medium from PTN plasmid transfected cells contained approximately 2 μg/ml PTN. Mammalian cells transfected with the PTN plasmid are therefore capable of producing and secreting PTN.

### Example 10

### PTN Plasmid Synthesize and Secret Functional PTN

It was then determined whether the secreted PTN was biologically active using a low attachment, anchorage independent growth assay. PTN, isolated from the media of 293 cells transfected with PTN plasmid, and a tetrazolium compound that is reduced by viable cells into a formazan product was added to each well containing SW13 cells. The amount of formazan, which directly correlates with the amount of proliferation, was measured by reading absorbance at 490 nm. The presence of PTN increased the amount of proliferation in a dose-dependent manner similar to basic fibroblast growth factor (bFGF) (FIG. 2b). Thus, mammalian cells transfected with the PTN plasmid are capable of synthesizing and secreting functional PTN protein.

### Example 11

### PTN Plasmid Induces Neovasculature Formation

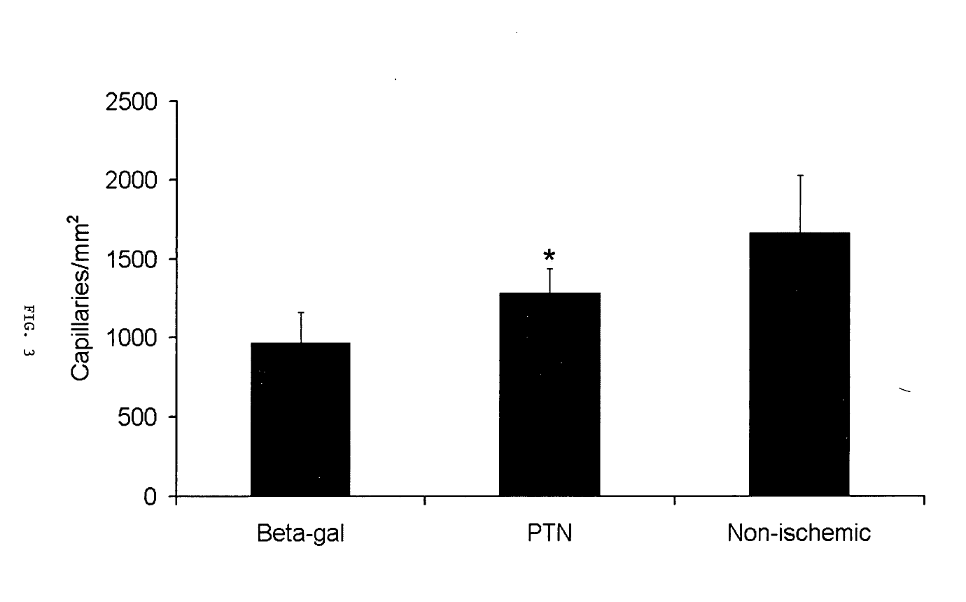

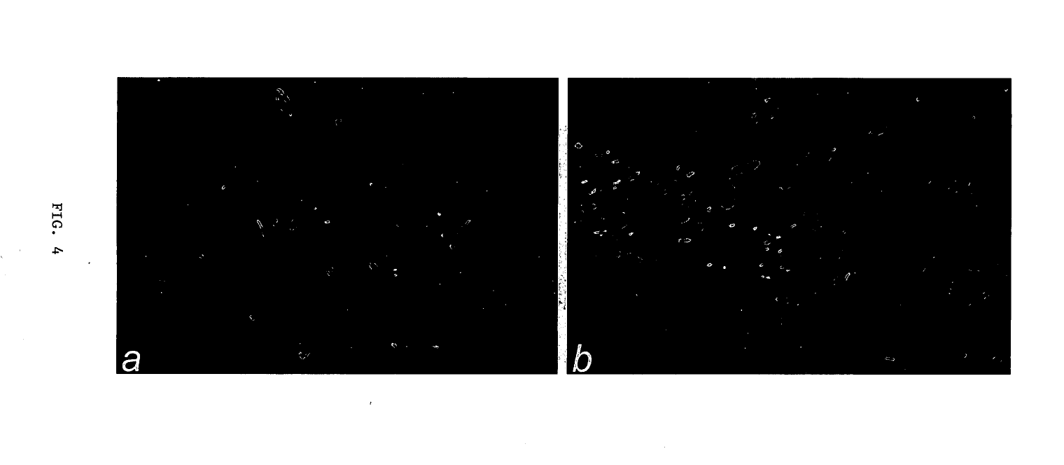

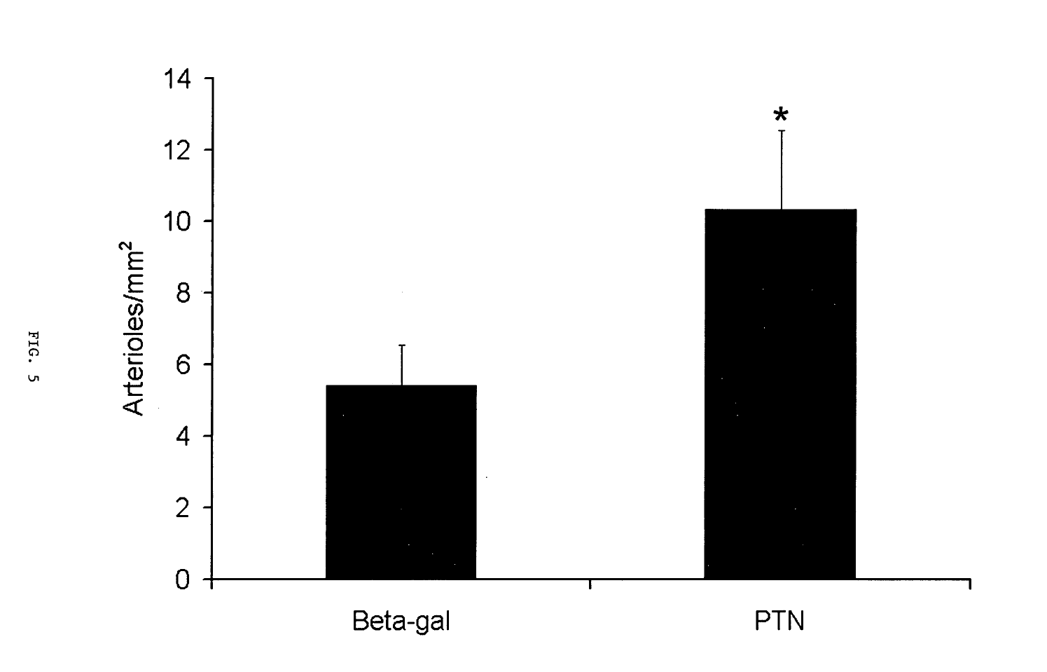

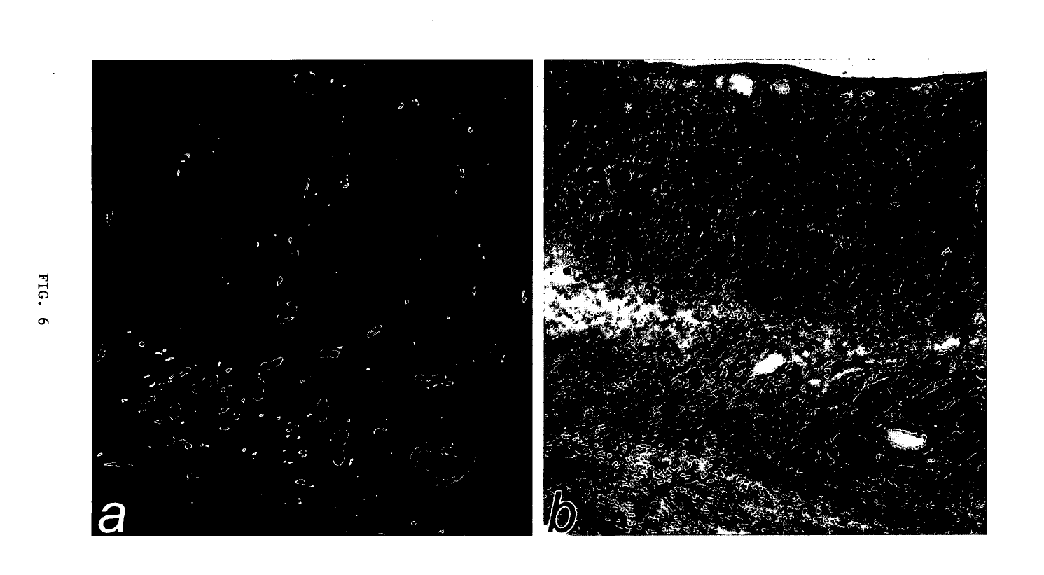

In order to show direct in vivo angiogenic activity of PTN in ischemic myocardium, it was determined if myocardial injection of naked PTN plasmid would stimulate the growth of new blood vessels in a rat myocardial ischemia reperfusion model. Five weeks following injection of plasmid, the capillary and arteriole density in the infarct scar was determined by immunostaining. The capillary density was significantly higher in animals injected with PTN plasmid compared to those injected with the control β-gal plasmid (P=0.02). Capillary density increased to 1287±148 capillaries per mm² after PTN plasmid injection compared to 970±195 capillaries per mm² after β-gal plasmid injection (FIG. 3). For reference, the capillary density of normal, non-ischemic myocardium was calculated as 1665±367 capillaries per mm². Injection of PTN plasmid also significantly increased arteriole density compared to injection of β-gal plasmid (P=0.002) (FIG. 4). Arteriole density increased to 10±2 arterioles per mm² in the presence of PTN compared to 5±1 arterioles per mm (FIG. 5). Upon histological examination, the increase in vessels appeared to be localized to the infarcted tissue (FIG. 6). Notably, there was also no macroscopic or histological evidence of angioma formation.

### Example 12

### PTN Plasmid Stimulates Growth of New Blood Vessels

An additional two rats were injected with PTN plasmid and sacrificed after 3 months. The arteriole and capillary densities were similar to those 5 weeks after PTN plasmid injection. The arteriole density after 3 months was 10±2 arterioles per mm² while the capillary density was 1289±209 capillaries per mm². Again, there was no macroscopic or histological evidence of vascular tumor formation.

### Example 13

### PTN-Stimulated New Vessels in Ischemic Myocardium are Functionally Connected to Existent Coronary Circulation

Six PTN-treated rats were first anesthetized with an intraperitoneal injection of 2001 μl of 50 mg/ml sodium pentobarbital and perfused with fluorescent microbeads. Briefly, the rats were injected with 700 μl of 50 μg/ml nitroglycerin in order to ensure vasodilation. After 10 minutes, the chest was opened and the abdominal aorta was cannulated with P-50 tubing. The left atrial appendage was cut to allow for drainage. 9 ml of saline was then perfused retrograde through the heart for approximately one minute. A 6 ml suspension of 0.2 μm fluorescent carboxylate-modified polystyrene beads (FluoSpheres, Molecular Probes) diluted 1:6 with PBS was then perfused through the heart. The hearts were immediately harvested, rinsed with PBS, and fresh frozen in O.C.T. freezing medium. They were then sectioned into 10 μm slices and examined under a Nikon TE 300 fluorescent microscope. Infarct areas were visualized by noting the paucity of microbeads in the area. New, PTN-induced vasculature in the infarcted myocardium was perfused with microbeads indicating that the vessels are functionally connected to the existent coronary circulation.

During normal development, PTN gene expression peaks during late embryogenesis and in perinatal growth. With the exception of a subset of neurons, PTN gene expression is markedly lower in adult tissues. Our results also demonstrate that PTN gene expression is upregulated in response to ischemia in rat myocardium, indicating a possible universal role in angiogenesis in response to ischemic conditions. PTN mRNA in both brain and myocardium is elevated beginning by day 3 following induction of ischemia and continues to be elevated in both tissues at two weeks. This temporal expression profile is noticeably different from the profile for VEGF and bFGF mRNA following myocardial ischemia. The VEGF mRNA expression profile follows a more acute response with elevated levels as early as 30 minutes after onset of ischemia.

Functionally, VEGF is thought to be involved in the initial phases of angiogenesis by affecting the permeability, recruitment and proliferation of endothelial cells. There is also an acute response with bFGF which is upregulated within 6 hours following ischemia. In contrast, the later expression profile of PTN indicates that it may play a more regenerative role influencing the maturation of the initial capillary beds into functional collateral arteries.

The results also indicate that injection of naked PTN plasmid in the myocardium induces neovasculature formation in ischemic myocardium, resulting in a higher capillary and arteriole density in the infarct scar. It is significant that injection of PTN plasmid results in both capillary and arteriole formation in ischemic myocardium since formation solely of capillaries does not necessarily result in a sustained increase in blood flow, due to the ease of regression of vessels without smooth muscle. In contrast, VEGF results solely in the formation of capillaries. Without the development of vessels with smooth muscle (e.g. arterioles), the newly formed capillary bed will fail to properly perfuse the ischemic tissue. It has also been shown that the capillary bed formed as a result of VEGF injection, either in plasmid form or secreted from retroviral transduced myoblasts, is irregular and not connected to the coronary vasculature. High doses of VEGF also produced angiomatous structures. In contrast, PTN plasmid was capable of inducing angiogenesis and arteriogenesis, without inducing vascular tumors. The vessel densities after both five weeks and 3 months following PTN plasmid injection were also similar, thus indicating that PTN has a sustained effect by forming long lasting, non-regressing vessels. The increase in vasculature as a result of PTN plasmid injection was also localized to the infarct tissue and not apparent in the surrounding normal myocardium. Importantly, the new vasculature in the ischemic zone was functionally connected with the existent coronary circulation indicating that not only can PTN stimulate the formation of blood vessels but the new vessels will provide sustained perfusion to an ischemic region and save viable myocardium and prevent further morbities associated with untreated infarcted myocardium. When compared to other agents such as VEGF, which has had detrimental side effects in pre-clinical studies, and both bFGF and VEGF, which have had mixed results in initial clinical trials, PTN appears to consistently produce the appropriate vasculature localized within ischemic myocardium. PTN may therefore be a potential therapeutic angiogenic agent for use in ischemic myocardium.

All of the references cited herein, including patents, patent applications, and publications, are hereby incorporated in their entireties by reference.

The present invention is not limited to the preferred embodiments, as it will be obvious to those of ordinary skill in the art that variations of the preferred embodiments may be used and that it is intended that the invention may be practiced otherwise than as specifically described herein. Accordingly, this invention includes all modifications encompassed within the spirit and scope of the invention as defined by the following claims.

The following is claimed:

1. A composition comprising pleiotrophin, wherein the composition induces or stimulates angiogenesis in a target tissue in vivo.

2. The composition of claim 1, wherein the target tissue is ischemic tissue.

3. The composition of claim 1, wherein the target tissue is cardiovascular tissue.

4. A vector comprising a promoter operably linked to a nucleic acid sequence encoding pleiotrophin, wherein the vector is capable of stimulating or inducing angiogenesis in vivo.

5. A method of inducing or stimulating angiogenesis in a target tissue in vivo, the method comprising administering to the target tissue a composition comprising pleiotrophin.

6. The method of claim 5, wherein the target tissue is cardiovascular tissue.

7. The method of claim 5, wherein the cardiovascular tissue is ischemic.

8. A method of inducing or stimulating angiogenesis in a target tissue in vivo, wherein the method comprises administering to the target tissue the vector of claim 4.

9. The method of claim 8, wherein the target tissue is cardiovascular tissue.

10. The method of claim 9, wherein the cardiovascular tissue is ischemic.

11. A method of enhancing or increasing blood flow to a target tissue, the method comprising administering to the target tissue a composition comprising pleiotrophin.

12. The method of claim 11, wherein the target tissue is ischemic.

13. The method of claim 12, wherein the target tissue is cardiovascular tissue.

14. A method of enhancing or increasing blood flow to a target tissue, the method comprising administering to the target tissue a composition comprising the vector of claim 4.

15. The method of claim 14, wherein the target tissue is ischemic.

16. The method of claim 15, wherein the target tissue is cardiovascular tissue.

17. A method of treating ischemic tissue, the method comprising administering to the ischemic tissue a composition comprising pleiotrophin.

18. The method of claim 17, wherein the ischemic tissue is in the heart.

19. A method of treating ischemic tissue, the method comprising administering to the ischemic tissue the vector of claim 4.

20. The method of claim 19, wherein the ischemic tissue is in the heart.

21. A method of preventing or reducing myocardial tissue damage, the method comprising administering to a subject in need a composition comprising pleiotrophin.

22. The method of claim 22, wherein the method induces or stimulates angiogenesis in myocardial tissue.

23. A method of preventing or reducing myocardial tissue damage, the method comprising administering to a subject in need the vector of claim 4.

24. The method of claim 23, wherein the vector induces or stimulates angiogenesis in myocardial tissue.

25. A method of preventing or treating a cardiovascular disease, the method comprising administering to a subject in need a composition comprising pleiotrophin.

26. The method of claim 25, wherein the cardiovascular disease comprises myocardial infarction, myocardial ischemia, angina pectoris, or cardiovascular tissue damage.

27. A method of preventing or treating a cardiovascular disease, the method comprising administering to a subject in need the vector of claim 4.

28. The method of claim 27, wherein the cardiovascular disease comprises myocardial infarction, myocardial ischemia, angina pectoris, or cardiovascular tissue damage.
